SEQUENCE LISTING (2 sequences)

SEQ ID NO: 1 - protein, 903 aa, Homo sapiens
MSDSGSQLGSMGSLTMKSQLQITVISAKLKENKKNWFGPSPYVEVTVDGQSKKTEKCNNTNSPKWKQPLTVIVTPVSKLHFRVWSHQTLKSDVLLGTAALDIYETLKSNNMKLEEVVVTLQLGGDKEPTETIGDLSICLDGLQLESEVVTNGETTCSENGVSLCLPRLECNSAISAHCNLCLPGLSDSPISASRVAGFTGASQNDDGSRSKDETRVSTNGSDDPEDAGAGENRRVSGNNSPSLSNGGFKPSRPPRPSRPPPPTPRRPASVNGSPSATSESDGSSTGSLPPTNTNTNTSEGATSGLIIPLTISGGSGPRPLNPVTQAPLPPGWEQRVDQHGRVYYVDHVEKRTTWDRPEPLPPGWERRVDNMGRIYYVDHFTRTTTWQRPTLESVRNYEQWQLQRSQLQGAMQQFNQRFIYGNQDLFATSQSKEFDPLGPLPPGWEKRTDSNGRVYFVNHNTRITQWEDPRSQGQLNEKPLPEGWEMRFTVDGIPYFVDHNRRTTTYIDPRTGKSALDNGPQIAYVRDFKAKVQYFRFWCQQLAMPQHIKITVTRKTLFEDSFQQIMSFSPQDLRRRLWVIFPGEEGLDYGGVAREWFFLLSHEVLNPMYCLFEYAGKDNYCLQINPASYINPDHLKYFRFIGRFIAMALFHGKFIDTGFSLPFYKRILNKPVGLKDLESIDPEFYNSLIWVKENNIEECDLEMYFSVDKEILGEIKSHDLKPNGGNILVTEENKEEYIRMVAEWRLSRGVEEQTQAFFEGFNEILPQQYLQYFDAKELEVLLCGMQEIDLNDWQRHAIYRHYARTSKQIMWFWQFVKEIDNEKRMRLLQFVTGTCRLPVGGFADLMGSNGPQKFCIEKVGKENWLPRSHTCFNRLDLPPYKSYEQLKEKLLFAIEETEGFGQE

SEQ ID NO: 2 - protein, 261 aa, Homo sapiens
MALNKNHSEGGGVIVNNTESILMSYDHVELTFNDMKNVPEAFKGTKKGTVYLTPYRVIFLSKGKDAMQSFMMPFYLMKDCEIKQPVFGANYIKGTVKAEAGGGWEGSASYKLTFTAGGAIEFGQRMLQVASQASRGEVPSGAYGYSYMPSGAYVYPPPVANGMYPCPPGYPYPPPPPEFYPGPPMMDGAMGYVQPPPPPYPGPMEPPVSGPDVPSTPAAEAKAAEAAASAYYNPGNPHNVYMPTSQPPPPPYYPPEDKKTQ

(19) United States
(12) Patent Application Publication
(10) Pub. No.: US 2017/0045520 A1
(43) Pub. Date: Feb. 16, 2017

(54) METHOD FOR PREDICTING RESPONSE TO THERAPY FOR CANCER

(71) Applicant: National University of Singapore, Singapore (SG)

(72) Inventor: Yoon Pin Lim, Singapore (SG)

(73) Assignee: National University of Singapore, Singapore (SG)

(21) Appl. No.: 15/305,905

(22) PCT Filed: Apr. 24, 2015
(86) PCT No.: PCT/SG2015/050085

§ 371 Date: Oct. 21, 2016

(30) Foreign Application Priority Data
Apr. 24, 2014 (SG) 10201401785R

(51) Int. Cl. G01N 33/574; A61K 38/53; C12Q 1/68

(52) U.S. Cl. CPC G01N 33/57496; C12Q 1/6886; A61K 38/53; C12Y 603/02019; G01N 2333/9015; G01N 2800/52

(57) ABSTRACT

The present invention provides a method of predicting response of a cancer patient to a therapy. The method comprises the step of determining a presence or absence of at least one mutation of ITCH (SEQ ID NO: 1) in a first sample isolated from a cancer patient, wherein the presence of a mutation is predictive of response of the cancer patient to a therapy selected from the group consisting of: Wnt pathway-, EGFR-, Her2-, hormonal- and WBP2-based therapy.

### CROSS-REFERENCE TO RELATED APPLICATION

This application claims the benefit of Singapore Patent Application No. 10201401785R filed on 24 Apr. 2014.

### FIELD OF THE INVENTION

The invention relates generally to methods and compositions for predicting response of a cancer patient to a therapy and/or for treatment of cancer and/or for prognosis of cancer.

### BACKGROUND TO THE INVENTION

The following discussion of the background to the invention is intended to facilitate an understanding of the present invention. However, it should be appreciated that the discussion is not an acknowledgment or admission that any of the material referred to was published, known or part of the common general knowledge in any jurisdiction as at the priority date of the application.

WW-binding protein 2 (WBP2) is a mediator of EGFR, ER and Wnt signalling in breast cancer cells. WBP2 and proteins that regulate its expression can be used to predict response to drugs that target EGFR, ER and Wnt signalling pathways. However, proteins that regulate WBP2 expression/activity are largely unknown. Thus, identifying interactors of WBP2 or regulators of WBP2 expression is desirable. Therefore, the present invention seeks to identify a regulator of WBP2 expression.

### SUMMARY OF THE INVENTION

In accordance with a first aspect of the invention, there is provided a method of predicting response of a cancer patient to a therapy, comprising the step of:

(i) determining a presence or absence of at least one mutation of ITCH (SEQ ID NO: 1) in a first sample isolated from a cancer patient,
wherein the presence of at least one mutation is predictive of response of the cancer patient to a therapy selected from the group consisting of Wnt pathway-, EGFR-, Her2, hormonal- and WBP2-based therapy, and the at least one mutation is selected, in any one or more, and in any combination, from the group consisting of E184K, E238K, E436D, E718K, L724V, E738Q, E746Q, R833C and E855K mutation of SEQ ID NO: 1.

ITCH refers to itchy E3 ubiquitin protein ligase homolog (SEQ ID NO: 1).

In relation to the group consisting of E184K, E238K, E436D, E718K, L724V, E738Q, E746Q, R833C and E855K mutation of SEQ ID NO: 1, the numbers flanked by the alphabets refer to the positions of the amino acids affected (counting from the N-terminus of the protein). The alphabet to the left of the numbers refer to the original (non-mutated) identity of the amino acids, the alphabet to the right of the numbers refer to the identity of the amino acid after mutation. E: glutamate, K: lysine, D: aspartate, L: leucine, V: valine, Q: glutamine, R: arginine, C: cysteine.

Preferably, the method further comprises the steps of:
(i) measuring an amount of polypeptide, mRNA or gene copy number of WBP2 (SEQ ID NO: 2) in the first sample isolated from the cancer patient; and
(ii) comparing the amount of polypeptide, mRNA or gene copy number of SEQ ID NO: 2 measured in the first sample to an amount of polypeptide of SEQ ID NO: 2 in a second sample isolated from normal cells,
wherein an increase in the amount of polypeptide, mRNA or gene copy number of SEQ ID NO: 2 measured in the first sample relative to the amount of polypeptide, mRNA or gene copy number of SEQ ID NO: 2 in the second sample is predictive of response of the breast cancer patient to a therapy selected from the group consisting of Wnt pathway-, EGFR-, Her2, hormonal- and WBP2-based therapy.

WBP2 refers to WW-binding protein 2 (SEQ ID NO: 2).

Preferably, the method further comprises the step of:
(i) sequencing the polypeptide of ITCH (SEQ ID NO: 1) in the sample isolated from the cancer patient, wherein ITCH is a regulator of WBP2 and the presence of the at least one mutation selected, in any one or more, and in any combination, from the group consisting of E184K, E238K, E436D, E718K, L724V, E738Q, E746Q, R833C and E855K mutation of SEQ ID NO: 1 is predictive of response of the cancer patient to a therapy selected from the group consisting of Wnt pathway-, EGFR-, Her2, hormonal- and WBP2-based therapy.

Preferably, ITCH downregulates the expression of WBP2 and that this is dependent on the E3 ligase activity of ITCH.

Preferably, ITCH modulates the WBP2-mediated Wnt pathway.

Preferably, an over-expression of ITCH decreases WBP2-mediated Wnt activation and an under-expression of ITCH increases WBP2-mediated Wnt activation.

Preferably, the at least one mutation of SEQ ID NO: 1 is E184K, R833C and/or E8555K.

Preferably, the at least one mutation of SEQ ID NO: 1 is E855K.

Preferably, the therapy is selected from the group consisting of Wnt pathway- and WBP2-based therapy.

Preferably, the cancer is selected from the group consisting of breast cancer and epithelial cancers.

In accordance with a second aspect of the invention, there is provided a composition comprising ITCH, wherein ITCH negatively regulates WBP2 in a cancer cell.

Preferably, ITCH downregulates the expression of WBP2 in a cancer cell.

Preferably, ITCH decreases WBP2-mediated Wnt activation by downregulating the expression of WBP2 in a cancer cell.

Preferably, the cancer is selected from the group consisting of breast cancer and epithelial cancers.

The cancer cell can be in vivo or in vitro. In one embodiment the cancer cell is in vivo. In another embodiment the cancer cell is in vitro.

In accordance with a third aspect of the invention, there is provided a method of selecting a cancer patient for a therapy, comprising the step of determining a presence or absence of at least one mutation of ITCH (SEQ ID NO: 1) in a sample isolated from a cancer patient, wherein the presence of at least one mutation in the sample is indicative that said cancer patient is suitable for a therapy selected from the group consisting of Wnt pathway-, EGFR-, Her2, hormonal- and WBP2-based therapy, and the at least one mutation is selected, in any one or more, and in any combination, from the group consisting of E184K, E238K, E436D, E718K, L724V, E738Q, E746Q, R833C and E855K mutation of SEQ ID NO: 1.

Preferably, the at least one mutation of SEQ ID NO: 1 is E184K, R833C and/or E855K.

Preferably, the at least one mutation of SEQ ID NO: 1 is E855K.

Preferably, the therapy is selected from the group consisting of Wnt pathway- and WBP2-based therapy.

Preferably, the cancer is selected from the group consisting of breast cancer and epithelial cancers.

In accordance with a fourth aspect of the invention, there is provided a method of treating a cancer patient, comprising the step of administering to the patient a composition comprising ITCH, wherein ITCH negatively regulates WBP2.

Preferably, the method further comprises the step of directing ITCH into the nucleus of a cancer cell.

Preferably, the cancer is selected from the group consisting of breast cancer and epithelial cancers

In accordance with a fifth aspect of the invention, there is provided a composition comprising ITCH for use in the treatment of cancer, wherein ITCH negatively regulates WBP2 in a cancer cell.

Preferably, ITCH downregulates the expression of WBP2 in a cancer cell.

Preferably, ITCH decreases WBP2-mediated Wnt activation by downregulating the expression of WBP2 in a cancer cell.

Preferably, the cancer is selected from the group consisting of breast cancer and epithelial cancers.

In accordance with a sixth aspect of the invention, there is provided a use of ITCH for the manufacture of a composition for the treatment of cancer, wherein ITCH negatively regulates WBP2 in a cancer cell.

Preferably, ITCH downregulates the expression of WBP2 in a cancer cell.

Preferably, ITCH decreases WBP2-mediated Wnt activation by downregulating the expression of WBP2 in a cancer cell.

Preferably, the cancer is selected from the group consisting of breast cancer and epithelial cancers.

Other aspects and advantages of the present invention will become apparent to those skilled in the art from a review of the ensuing description, which proceeds with reference to the following illustrative drawings of preferred embodiments.

Sequences of ITCH and WBP2

Sequence of ITCH (SEQ ID NO: 1):
MSDSGSQLGS MGSLTMKSQL QITVISAKLK ENKKNWEGPS
PYVEVTVDGQ SKKTEKCNNT NSPKWKQPLT VIVTPVSKLH
FRVWSEQTLK SDVLLGTAAL DIYETLKSNN MKLEEVVVTL
QLGGDKEPTE TIGDLSICLD GLQLESEVVT NGETTCSENG
VSLCLPRLEC NSAISARCNL CLPGLSDSPI SASRVAGFTG
ASQNDDGSRS KDETRVSTNG SDDPEDAGAG ENRRVSGNNS
PSLSNGGFKP SRPPRPSRPP PPTPRRPASV NGSPSATSES
DGSSTGSLPP TNTNTNTSEG ATSGLIIPLT ISGGSGPRPL
NPVTQAPLPP GWEQRVDQHG RVYYVDHVEK RTTWDRPEPL
PPGWERRVDN MGRIYYVDHF TRTTTWQRPT LESVRNYEQW
QLQRSQLQGA MQQFNQRFIY GNQDLFATSQ SKEFDPLGPL
PPGWEKRTDS NGRVYFVNHN TRITQWEDPR SQGQLNEKPL
PEGWEMRFTV DGIPYFVDEN RRTTTYIDPR TGKSALDNGP
QTAYVRDFKA KVQYFRFWCQ QLAMPQHIKI TVTRKTLFED
SFQQIMSFSP QDLRRRLWVI FPGEEGLDYG GVAREWFFLL
SHEVLNPMYC LFEYAGKDNY CLQINPASYI NPDHLKYFRF
IGRFIAMALF HGKFIDTGES LPFYKRILNK PVGLKDLESI
DPEFYNSLIW VKENNIEECD LEMYFSVDKE ILGEIKSHDL
KPNGGNILVT EENKEEYIRM VAEWRLSRGV EEQTQAFFEG
FNEILPQQYL QYFDAKELEV LLCGMQEIDL NDWQRHAIYR
HYARTSKQIM WFWQFVKEID NEKRMRLLQF VTGTCRLPVG
GFADLMGSNG PQKFCIEKVG KENWLPRSHT CFNRLDLPPY
KSYEQLKEKL LFAIEETEGF GQE
Sequence of WBP2 (SEQ ID NO: 2):
MALNKNHSEG GGVIVNNTES ILMSYDHVEL TFNDMKNVPE
AFKGTKKGTV YLTPYRVIFL SKGKDAMQSF MMPFYLMKDC
EIKQPVFGAN YIKGTVKAEA GGGWEGSASY KLTFTAGGAI
EFGQRMLQVA SQASRGEVPS GAYGYSYMPS GAYVYPPPVA
NGMYPCPPGY PYPPPPPEFY PGPPMMDGAM GYVQPPPPPY
PGPMEPPVSG PDVPSTPAAE AKAAEAAASA YYNPGNPHNV
YMPTSQPPPP PYYPPEDKKT Q

### BRIEF DESCRIPTION OF THE DRAWINGS

Preferred embodiments of the invention will be described, by way of illustrative examples only, with reference to the following drawings, of which:

FIG. 1: Yeast-2-hydrid screening through which ITCH was found to interact with WBP2.

FIG. 2: Interaction of endogenous (A) and exogenous (B) WBP2 and ITCH via co-immunoprecipitation.

FIG. 3: A: ITCH overexpression targets endogenous WBP2 for protein degradation. B: Tyrosine phosphorylation of WBP2 interfered with ITCH-mediated downregulation of WBP2 expression. E: Downreguation of WBP2 as a result of ITCH co-expression was due to proteosome mediated degradation.

FIG. 4: Downregulation of WBP2 by ITCH has a negative effect on the Wnt pathway. ITCH overexpression significantly abolished and potentiated WBP2/β-catenin-mediated Wnt pathway activation.

FIG. 5: Shows the distribution of mutations on ITCH protein. C2: Calcium binding domain; PRD: Proline rich domain; WW: Trytophan-Tryptophan domain; HECT: Homologus to E6-AP Carboxy Terminus (contains E3 ligase catalytic site).

FIG. 6: Overexpression of mutant ITCH protein along with WBP2 result in a lesser downregulation of WBP2 compared to ITCH WT.

FIG. 7: ITCH mutants result in diminished inhibitory effect on WBP2-mediated Wnt activation compared to ITCH WT.

FIG. 8: ITCH-mediated WBP2 degradation regulates drug sensitivity to Wnt inhibitor-059.

FIG. 9: ITCH-mediated WBP2 degradation regulates drug sensitivity to Wnt Inhibitor-FH535.

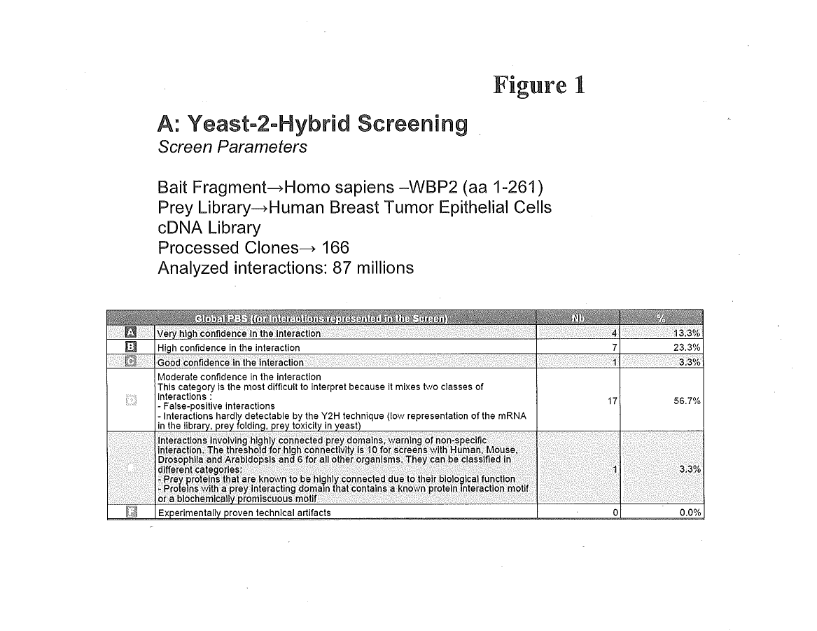

Other arrangements of the invention are possible and, consequently, the accompanying drawings are not to be understood as superseding the generality of the preceding description of the invention.

### PREFERRED EMBODIMENTS OF THE INVENTION

Particular embodiments of the present invention will now be described with reference to the accompany drawings. The terminology used herein is for the purpose of describing particular embodiments only and is not intended to limit the scope of the present invention. Additionally, unless defined otherwise, all technical and scientific terms used herein have the same meanings as commonly understood by one or ordinary skill in the art to which this invention belongs.

The present technology relates to the interaction of WW-binding protein 2 (WBP2) with itchy E3 ubiquitin protein ligase homolog (ITCH), the effect of their interactions in terms of WBP2 expression and Wnt pathway activity as well as the rest of the information associated with WBP2 and ITCH described hereinafter are novel and have not been reported.

The advantages of the present technology include at least the following:—
Use of ITCH mutation as a biomarker or companion biomarker to select cancer patients for Writ pathway-, EGFR-, Her2, hormonal- and WBP2-based therapy. Preferably, to use ITCH mutation as a biomarker or companion biomarker to select cancer patients for WBP2 or Wnt-based therapy. The type of cancer includes at least breast cancer and epithelial cancers like gastric cancer, colon cancer, ovarian cancer, prostate cancer and the like.
High ITCH expression or absence of its mutation and the absence or low expression of WBP2 can be a dual biomarker system for good prognosis of cancer such as breast cancer and epithelial cancers like gastric cancer, colon cancer, ovarian cancer, prostate cancer, and the like.

In accordance with an aspect of the invention, there is disclosed hereinafter a method of predicting response of a cancer patient to a therapy, comprising the step of determining a presence or absence of at least one mutation of ITCH (SEQ ID NO: 1) in a first sample isolated from a cancer patient, wherein the presence of at least one mutation is predictive of response of the cancer patient to a therapy selected from the group consisting of Wnt pathway-, EGFR-, Her2, hormonal- and WBP2-based therapy, and the at least one mutation is selected, in any one or more, and in any combination, from the group consisting of E184K, E238K, E436D, E718K, L724V, E738Q, E746Q, R833C and E855K mutation of SEQ ID NO: 1.

In one embodiment, the cancer is breast cancer.

In another embodiment, the cancer is an epithelial cancer such as gastric cancer, colon cancer, ovarian cancer, prostate cancer and the like.

In relation to the group consisting of E184K, E238K, E436D, E718K, L724V, E738Q, E746Q, R833C and E855K mutation of SEQ ID NO: 1, the numbers flanked by the alphabets refer to the positions of the amino acids affected (counting from the N-terminus of the protein). The alphabet to the left of the numbers refer to the original (non-mutated) identity of the amino acids, the alphabet to the right of the numbers refer to the identity of the amino acid after mutation. E: glutamate, K: lysine, D: aspartate, L: leucine, V: valine, Q: glutamine, R: arginine, C: cysteine.

The method further comprises the steps of (i) measuring an amount of polypeptide, mRNA or gene copy number WBP2 (SEQ ID NO: 2) in the first sample isolated from the cancer patient; and (ii) comparing the amount of polypeptide, mRNA or gene copy number of SEQ ID NO: 2 measured in the first sample to an amount of polypeptide of SEQ ID NO: 2 in a second sample isolated from normal cells, wherein an increase in the amount of polypeptide, mRNA or gene copy number of SEQ ID NO: 2 measured in the first sample relative to the amount of polypeptide, mRNA or gene copy number of SEQ ID NO: 2 in the second sample is predictive of response of the cancer patient to a therapy selected from the group consisting of Wnt pathway-, EGFR-, Her2, hormonal- and WBP2-based therapy.

In one embodiment, the cancer is breast cancer.

In another embodiment, the cancer is an epithelial cancer such as gastric cancer, colon cancer, ovarian cancer, prostate cancer and the like.

The method also comprises the step of sequencing the polypeptide of ITCH (SEQ ID NO: 1) in the sample isolated from the cancer patient, wherein ITCH is a regulator of WBP2 and the presence of the at least one mutation selected, in any one or more, and in any combination, from the group consisting of E184K, E238K, E436D, E718K, L724V, E738Q, E746Q, R833C and E855K mutation of SEQ ID NO: 1 is predictive of response of the cancer patient to a therapy selected from the group consisting of Wnt pathway-, EGFR-, Her2, hormonal- and WBP2-based therapy.

In one embodiment, the cancer is breast cancer.

In another embodiment, the cancer is an epithelial cancer such as gastric cancer, colon cancer, ovarian cancer, prostate cancer and the like.

Preferably, ITCH down regulates the expression of WBP2 and that this is dependent on the E3 ligase activity of ITCH.

Preferably, ITCH modulates the WBP2-mediated Wnt pathway.

Preferably, an over-expression of ITCH decreases WBP2-mediated Writ activation and an under-expression of ITCH increases WBP2-mediated Writ activation.

The at least one mutation of ITCH can be any one or more, and in any combination, of the mutations set forth in the group consisting of E184K, E238K, E436D, E718K, L724V, E738Q, E7460, R833C and E855K mutation of SEQ ID NO: 1.

With reference to the method described above, it is preferable that the at least one mutation of SEQ ID NO: 1 is E184K, R833C and/or E855K. In one embodiment, the mutation is E184K of SEQ ID NO: 1. In another embodiment, the mutation is R833C of SEQ ID NO: 1. In another embodiment, the mutation is E855K of SEQ ID NO: 1. In another embodiment, the mutations are E184K and R833C of SEQ ID NO: 1. In another embodiment, the mutations are E184K and E855K of SEQ ID NO: 1. In another embodiment, the mutations are E833C and E855K of SEQ ID NO: 1. In another embodiment, the mutations are E184K, R833C and E855K of SEQ ID NO: 1.

More preferably, the at least one mutation of SEQ ID NO: 1 is E855K.

Preferably, the therapy is selected from the group consisting of Wnt pathway- and WBP2-based therapy.

In accordance with another aspect of the invention, there is disclosed a composition comprising ITCH, wherein ITCH negatively regulates WBP2 in a cancer cell.

Preferably, ITCH downregulates the expression of WBP2 in a cancer cell. Preferably, ITCH decreases WBP2-mediated Wnt activation by downregulating the expression of WBP2 in a cancer cell.

In one embodiment the cancer cell is in vivo. In another embodiment the cancer cell is in vitro.

In one embodiment, the cancer is breast cancer.

In another embodiment, the cancer is an epithelial cancer such as gastric cancer, colon cancer, ovarian cancer, prostate cancer and the like.

In accordance with another aspect of the invention, there is disclosed a method of selecting a cancer patient for a therapy, comprising the step of determining a presence or absence of at least one mutation of ITCH (SEQ ID NO: 1) in a sample isolated from a cancer patient, wherein the presence of at least one mutation in the sample is indicative that said cancer patient is suitable for a therapy selected from the group consisting of Wnt pathway-, EGFR-, Her2, hormonal- and WBP2-based therapy, and the at least one mutation is selected, in any one or more, and in any combination, from the group consisting of E184K, E238K, E436D, E718K, L724V, E738Q, E746Q, R833C and E855K mutation of SEQ ID NO: 1.

The at least one mutation of ITCH can be any one or more, and in any combination, of the mutations set forth in the group consisting of E184K, E238K, E436D, E718K, L724V, E738Q, E746Q, R833C and E855K mutation of SEQ ID NO: 1.

With reference to the method described above, it is preferable that the at least one mutation of SEQ ID NO: 1 is E184K, R833C and/or E855K. In one embodiment, the mutation is E184K of SEQ ID NO: 1. In another embodiment, the mutation is R833C of SEQ ID NO: 1. In another embodiment, the mutation is E855K of SEQ ID NO: 1. In another embodiment, the mutations are E184K and R833C of SEQ ID NO: 1. In another embodiment, the mutations are E184K and E855K of SEQ ID NO: 1. In another embodiment, the mutations are E833C and E855K of SEQ ID NO: 1. In another embodiment, the mutations are E184K, R833C and E855K of SEQ ID NO: 1.

More preferably, the at least one mutation of SEQ ID NO: 1 is E855K.

Preferably, the therapy is selected from the group consisting of Wnt pathway- and WBP2-based therapy.

In one embodiment, the cancer is breast cancer.

In another embodiment, the cancer is an epithelial cancer such as gastric cancer, colon cancer, ovarian cancer, prostate cancer and the like.

In accordance with another aspect of the invention, there is disclosed a method of treating a cancer patient, comprising the step of administering to the patient a composition comprising ITCH, wherein ITCH negatively regulates WBP2.

Preferably, the method further comprises the step of directing ITCH into the nucleus of a cancer cell.

In one embodiment, the cancer is breast cancer.

In another embodiment, the cancer is an epithelial cancer such as gastric cancer, colon cancer, ovarian cancer, prostate cancer and the like.

In accordance with another aspect of the invention, there is disclosed a composition comprising ITCH for use in the treatment of cancer, wherein ITCH negatively regulates WBP2 in a cancer cell. Preferably, ITCH downregulates the expression of WBP2 in a cancer cell. Preferably, ITCH decreases WBP2-mediated Wnt activation by downregulating the expression of WBP2 in a cancer cell.

In one embodiment, the cancer is breast cancer.

In another embodiment, the cancer is an epithelial cancer such as gastric cancer, colon cancer, ovarian cancer, prostate cancer and the like.

In accordance with another aspect of the invention, there is disclosed use of ITCH for the manufacture of a composition for the treatment of cancer, wherein ITCH negatively regulates WBP2 in a cancer cell. Preferably, ITCH downregulates the expression of WBP2 in a cancer cell.

Preferably, ITCH decreases WBP2-mediated Wnt activation by downregulating the expression of WBP2 in a cancer cell.

In one embodiment, the cancer is breast cancer.

In another embodiment, the cancer is an epithelial cancer such as gastric cancer, colon cancer, ovarian cancer, prostate cancer and the like.

WBP2 interactors were identified via yeast-2-hybrid. The screening parameters include the following:—
Bait fragment: Homo sapiens—WBP2 (amino acid (or “aa”) 1-261)
Prey Library: Human breast tumour epithelial cells
cDNA library
Number of proceed clones: 166
Number of analysed interactions: 87 million

ITCH was found to interact with WBP2 through the yeast-2-hybrid screening.

Results

Interaction of Endogenous WBP2 and ITCH Via Co-Immunoprecipitation

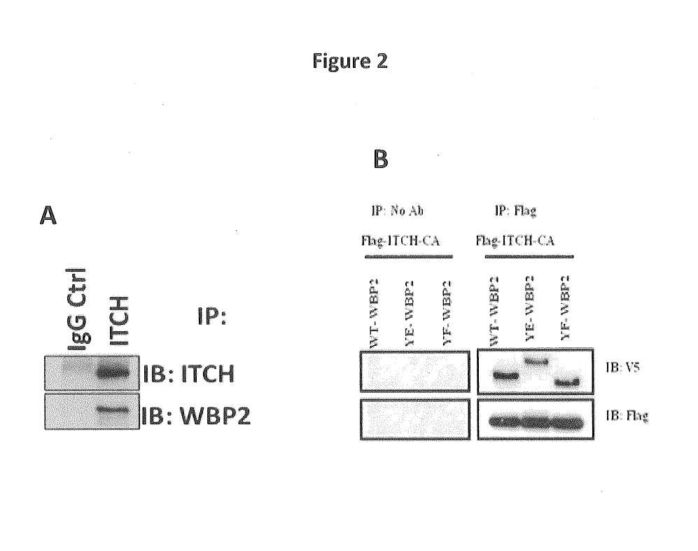

The interaction of endogenous WBP2 and ITCH via co-immunoprecipitation was validated (FIG. 2A). We demonstrated that tyrosine phosphorylation of WBP2 at Y192 and Y231 interfered with the binding between exogenous ITCH and WBP2 (FIG. 2B).

ITCH Overexpression Targets Endogenous WBP2 for Protein Degradation

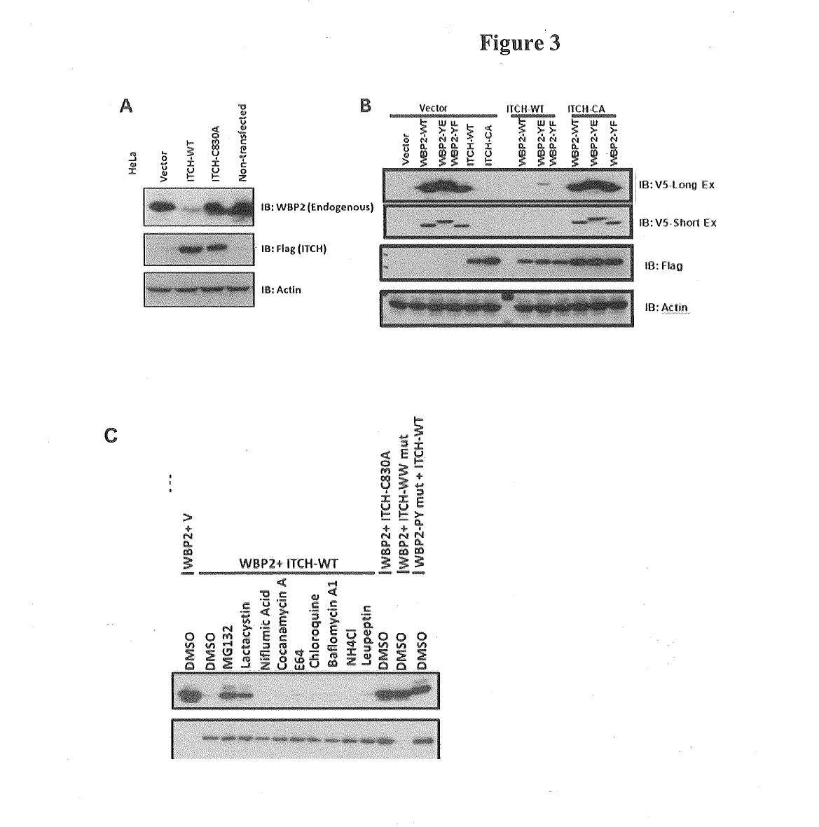

We demonstrated that ITCH Overexpression Targets Endogenous WBP2 for Protein Degradation (FIG. 3A). We also showed that tyrosine Phosphorylation of WBP2 interfered with ITCH-mediated downregulation of exogenous WBP2 expression (FIG. 3B). Our results revealed that downreguation of WBP2 as a result of ITCH co-expression was due to proteosome mediated degradation (FIG. 3C).

Downregulation of WBP2 by ITCH has a Negative Effect on the Wnt Pathway

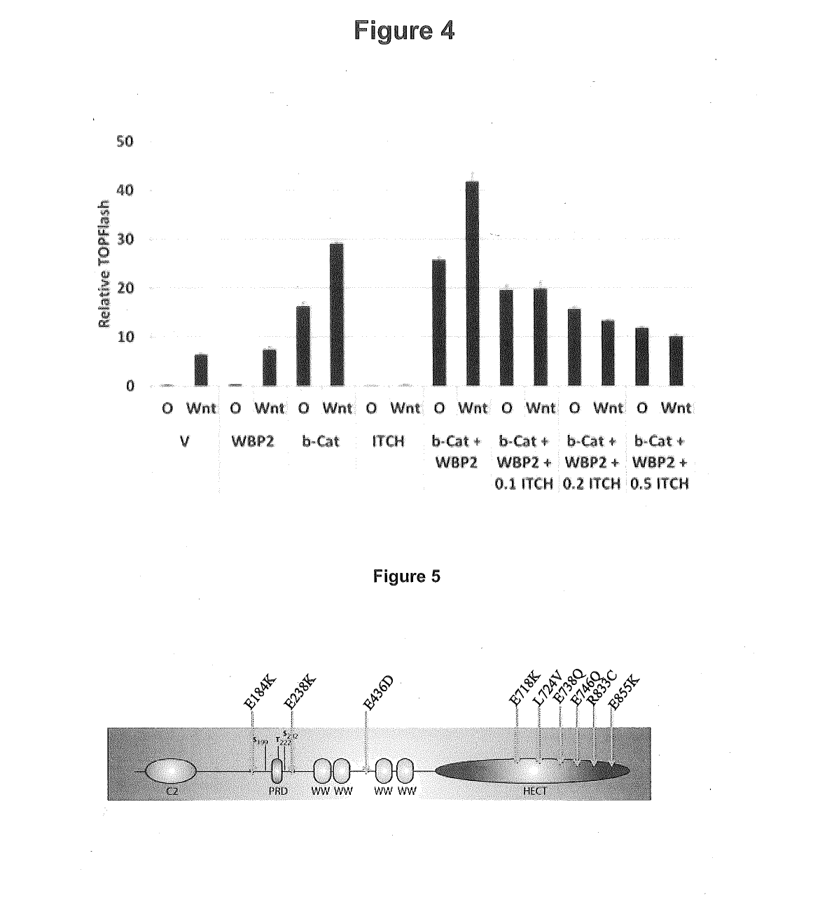

We further demonstrate that the downregulation of WBP2 by ITCH has a negative effect on the Wnt pathway. For example, ITCH Overexpression significantly abolished (FIG. 4) WBP2/β-catenin-mediated Wnt pathway activation

Mutations Resulting Loss of Function in ITCH

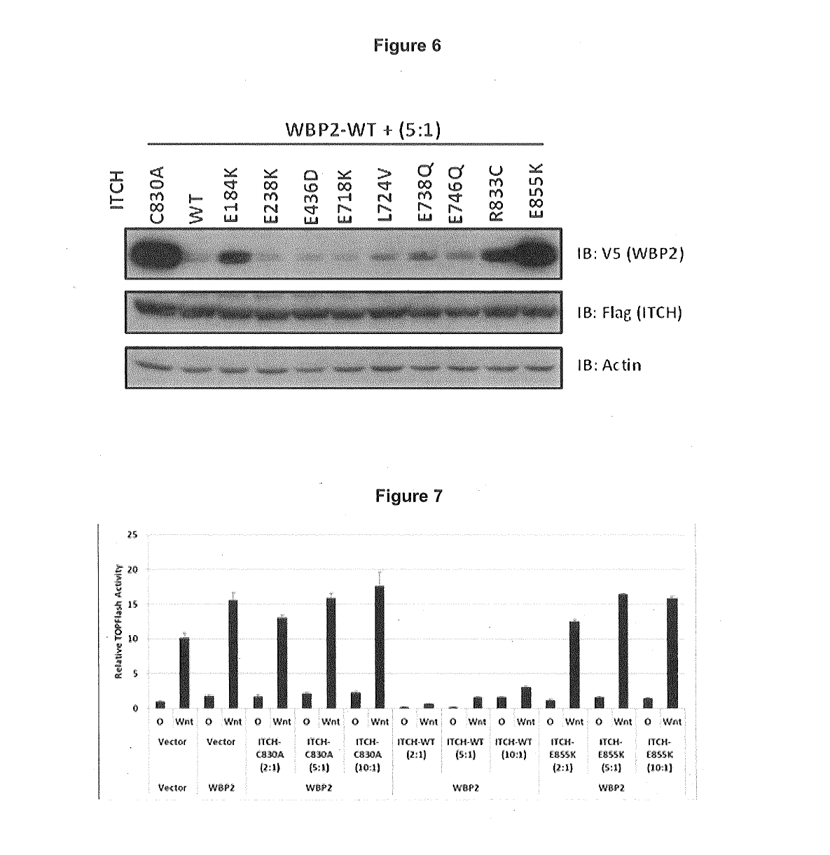

Clinical breast cancer tissues were found to possess ITCH mutations (see FIG. 5). Interestingly, majority of the mutations occur in the HECT domain, which is catalytic domain of the E3 ligase. We hypothesize that these mutations will result in a loss of function in ITCH. If true, overexpression of these mutants along with WBP2 should result in a lesser downregulation of WBP2 compared to ITCH WT. This was indeed proven to the case, especially R833C and E855K mutants (FIG. 6). ITCH CA refers to catalytically dead ITCH mutant.

Since overexpression of WT ITCH decreased WBP2 mediated Wnt activation, (FIG. 4), ITCH mutants are likely to result in diminished inhibitory effect on WBP2-mediated Wnt activation. Indeed, FIG. 7 supported this notion.

ITCH Mutation Sensitises WBP2 Overexpressing Cancer Cells to Wnt Inhibitor

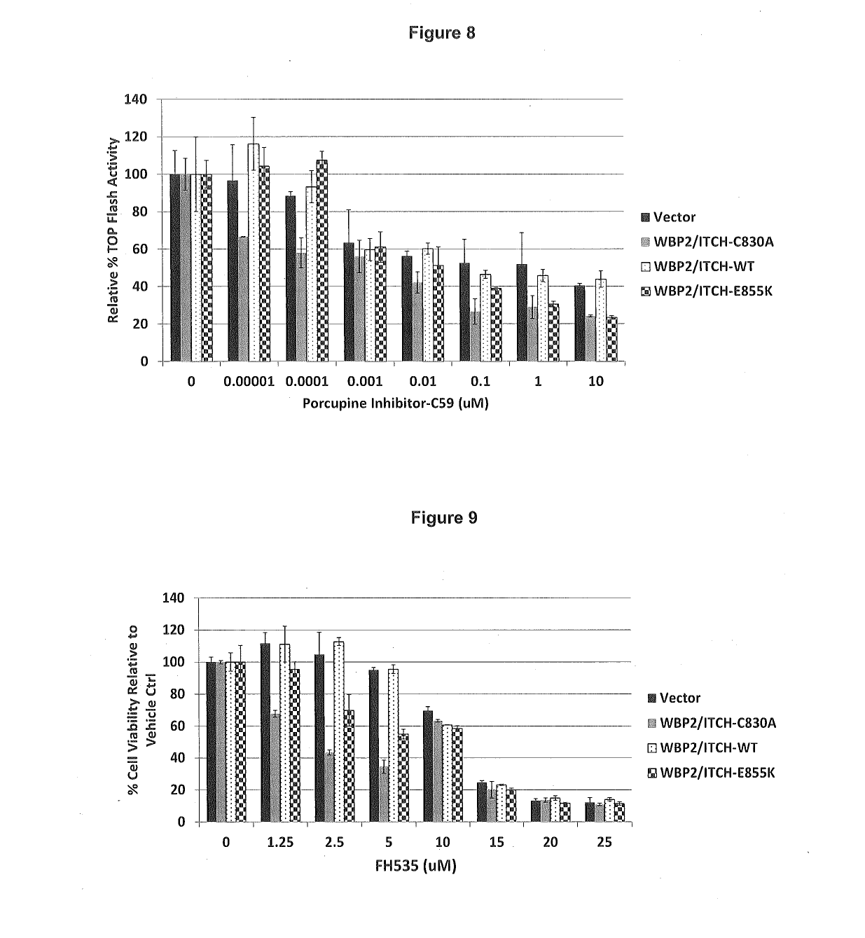

We tested the hypothesis that cells would be less sensitive to FH535 and C59, both inhibitors of Wnt signalling, when WT ITCH is overexpressed (ie., low WBP2) compared to when C830A or E855K mutant is overexpressed (i.e., high WBP2). This was indeed the case for up to 0.0001 μM and 5 μM for FH535 and C59, respectively, but above these doses, the distinction was lost probably as a result of non-specific cytotoxicity (FIGS. 8 and 9). This implies that ITCH inactivating mutations could be used to predict for response to Wnt inhibitors especially in cancers that are dependent on WBP2.

Applications of ITCH Mutation

Applications of ITCH mutation include at least the following:—
ITCH mutation can be used as a companion biomarker to select cancer patients for WBP2 or WBP2-dependent Wnt-based therapy. With reference to FIGS. 8 and 9, ITCH mediated regulation of WBP2 expression can influence sensitivity of cells to 2 different types of Wnt inhibitors (C59 and FH535). Currently, there is no FDA approved drug against the Wnt pathway. However, we expect FDA approval of Wnt inhibitors to happen within the next few years since Wnt pathway is a very critical oncogenic target for treatment of human cancers.
The high expression of ITCH or the absence of its mutation and the absence or low expression of WBP2 can be used as a dual biomarker system for good prognosis of cancer.
Drive ITCH into the nucleus to inhibit WBP2-dependent cancers.
To target cancer more effectively through dual inhibition of WBP2 and components of the Wnt pathway in ITCH-mutation positive cancer.
The type of cancer includes at least breast cancer and epithelial cancers like gastric cancer, colon cancer, ovarian cancer, prostate cancer and the like.
The frequency of ITCH mutation is higher in the aggressive breast cancer (Her2+ and triple negative breast cancer) compared to less aggressive ones (see Table 1 below). Hence, the Her2+ and TNBC subtypes of breast cancers will be the indication for the use of the above strategies in one application.

TABLE 1
Frequencies of ITCH mutations in clinical breast cancer.

| Histological | Mutations/AA | Mutation | |
|---|---|---|---|
| Subtype | Change | Frequency | Remarks |
| NS | E184K, E238K, | 0.35% (3/867) | E718K and E738Q |
| | E718K, E738Q | | were both found in |
| | | | the same sample |
| HER2+ | L724V, E746Q, | 5.2% (3/58) | |
| | L247L* | | |
| Basal | E436D, R833C, | 2.5% (3/122) | |
| (TNBC) | E855K | | |
| ER+/PR+ | NIL | 0% (0/65) | |

Source: COSMIC
NS: not specified.
*not studied.

As WBP2 and ITCH are ubiquitously expressed, it is conceivable that the relationship between ITCH, WBP2 and Wnt that was observed in breast cancer would also apply to other cancers such as epithelial cancers like gastric cancer, colon cancer, ovarian cancer, prostate cancer and the like.

Those skilled in the art will appreciate that the invention described herein is susceptible to variations and modifications other than those specifically described. The invention includes all such variation and modifications. The invention also includes all of the steps, features, formulations and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

Any manufacturer's instructions, descriptions, product specifications, and product sheets for any products mentioned herein or in any document incorporated by reference herein, are hereby incorporated herein by reference, and may be employed in the practice of the invention.

The present invention is not to be limited in scope by any of the specific embodiments described herein. These embodiments are intended for the purpose of exemplification only. Functionally equivalent products, formulations and methods are clearly within the scope of the invention as described herein.

The invention described herein may include one or more range of values (e.g. size, concentration, etc). A range of values will be understood to include all values within the range, including the values defining the range, and values adjacent to the range which lead to the same or substantially the same outcome as the values immediately adjacent to that value which defines the boundary to the range.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

1. A method of predicting response of a cancer patient to a therapy, comprising the step of:
(i) determining a presence or absence of at least one mutation of ITCH (SEQ ID NO: 1) in a first sample isolated from a cancer patient, wherein the presence of at least one mutation is predictive of response of the cancer patient to a therapy selected from the group consisting of Wnt pathway-, EGFR-, Her2, hormonal- and WBP2-based therapy, and the at least one mutation is selected, in any one or more, and in any combination, from the group consisting of E184K, E238K, E436D, E718K, L724V, E738Q, E746Q, R833C and E855K mutation of SEQ ID NO: 1.

2. The method of claim 1, further comprising the steps of:
(i) measuring an amount of polypeptide, mRNA or gene copy number WBP2 (SEQ ID NO: 2) in the first sample isolated from the cancer patient; and
(ii) comparing the amount of polypeptide, mRNA or gene copy number of SEQ ID NO: 2 measured in the first sample to an amount of polypeptide of SEQ ID NO: 2 in a second sample isolated from normal cells,
wherein an increase in the amount of polypeptide, mRNA or gene copy number of SEQ ID NO: 2 measured in the first sample relative to the amount of polypeptide, mRNA or gene copy number of SEQ ID NO: 2 in the second sample is predictive of response of the cancer patient to a therapy selected from the group consisting of Wnt pathway-, EGFR-, Her2, hormonal- and WBP2-based therapy.

3. The method of claim 1, further comprising the step of:
(i) sequencing the polypeptide of ITCH (SEQ ID NO: 1) in the sample isolated from the cancer patient, wherein ITCH is a regulator of WBP2 and the presence of the at least one mutation selected, in any one or more, and in any combination, from the group consisting of E184K, E238K, E436D, E718K, L724V, E738Q, E746Q, R833C and E855K mutation of SEQ ID NO: 1 is predictive of response of the cancer patient to a therapy selected from the group consisting of Wnt pathway-, EGFR-, Her2, hormonal- and WBP2-based therapy.

4. The method of claim 3, wherein ITCH downregulates the expression of WBP2 and that this is dependent on the E3 ligase activity of ITCH.

5. The method of claim 3, wherein ITCH modulates the WBP2-mediated Wnt pathway, and wherein an over-expression of ITCH decreases WBP2-mediated Wnt activation and an under-expression of ITCH increases WBP2-mediated Wnt activation.

6. (canceled)

7. The method of claim 1, wherein the at least one mutation of SEQ ID NO: 1 is E184K, R833C and/or E855K.

8. The method of claim 5, wherein the at least one mutation of SEQ ID NO: 1 is E855K.

9. The method of claim 1, wherein the therapy is selected from the group consisting of Wnt pathway- and WBP2-based therapy.

10. The method of claim 1, wherein the cancer is selected from the group consisting of breast cancer and epithelial cancers.

11. A composition comprising ITCH, wherein ITCH negatively regulates WBP2 in a cancer cell.

12. The composition of claim 11, wherein ITCH downregulates the expression of WBP2 in a cancer cell, and wherein ITCH decreases WBP2-mediated Wnt activation by downregulating the expression of WBP2 in the cancer cell.

13. (canceled)

14. The composition of claim 11, wherein the cancer is selected from the group consisting of breast cancer and epithelial cancers, and wherein the cancer cell is in vitro.

15. (canceled)

16. A method of selecting a cancer patient for a therapy, comprising the step of determining a presence or absence of at least one mutation of ITCH (SEQ ID NO: 1) in a sample isolated from a cancer patient, wherein the presence of at least one mutation in the sample is indicative that said cancer patient is suitable for a therapy selected from the group consisting of Wnt pathway-, EGFR-, Her2, hormonal- and WBP2-based therapy, and the at least one mutation is selected, in any one or more, and in any combination, from the group consisting of E184K, E238K, E436D, E718K, L724V, E738Q, E746Q, R833C and E855K mutation of SEQ ID NO: 1.

17. The method of claim 16, wherein the at least one mutation of SEQ ID NO: 1 is E184K, R833C and/or E855K.

18. The method of claim 17, wherein the at least one mutation of SEQ ID NO: 1 is E855K.

19. The method of claim 16, wherein the therapy is selected from the group consisting of Wnt pathway- and WBP2-based therapy.

20. The method of claim 16, wherein the cancer is selected from the group consisting of breast cancer and epithelial cancers.

21. A method of treating a cancer patient, comprising the step of administering to the patient a composition comprising ITCH, wherein ITCH negatively regulates WBP2.

22. The method of claim 21, further comprising the step of directing ITCH into the nucleus of a cancer cell.

23. The method of claim 21, wherein the cancer is selected from the group consisting of breast cancer and epithelial cancers.

24.-31. (canceled)
